(19) United States
(12) Patent Application Publication
(10) Pub. No.: US 2005/0130255 A1
(43) Pub. Date: Jun. 16, 2005

(54) Process control based on microbiological activity

(76) Inventors: Roy Christiaan Montijn, Amsterdam (NL); Frank Henri Johan Schuren, Veenendaal (NL)

Correspondence Address: NORRIS, MCLAUGHLIN & MARCUS, P.A., 875 THIRD AVE, 18TH FLOOR, NEW YORK, NY, 10022, US

(21) Appl. No.: 10/511,496

(22) PCT Filed: Apr. 8, 2003
(86) PCT No.: PCT/NL03/00264

(30) Foreign Application Priority Data
Apr. 17, 2002 (NL) 1020415

(51) Int. Cl. C12Q 1/04

(52) U.S. Cl. 435/34

(57) ABSTRACT

The present invention relates to the determination of environmental conditions for the purpose of process control using microorganisms. The present invention uses the priciple that intrinsic changes in a microorganism after applying an external stimulus are characteristic of the nature of the stimulus the microorganism receives from outside. By measuring these intrinsic changes, microorganisms can be used as a specific sensor to determine environmental conditions, to determine the degree of change in these environmental conditions, to determine the nature and force of the stimulus and to be able to measure the effects of a diversity of stimuli on various processes an thus control these processes.

The invention relates to the determination of environmental conditions. In particular, the present invention relates to the determination of environmental conditions for the purpose of process control using microbiological microorganisms.

Many industrial and natural processes, for example the preparation and packaging of food, cannot be properly controlled because the prevailing process conditions are not easy to determine. As a result of this absence of knowledge concerning the prevailing process conditions, control thereof is very difficult and mostly inefficient.

In many cases, the processes are determined or to a large extent affected by the desired or undesired presence of microorganisms. Examples of such processes are food preparation processes, food packaging, biofilm formation processes, fermentation processes and bioconversion processes.

Control of the activity of the microorganisms that play a role in these processes, such as killing off undesired microorganisms, or conversely, stimulating the activity of desired microorganisms, is often problematic. The effectiveness of the process of killing off undesired microorganisms, for example, is traditionally done by determining afterwards the number of still surviving microorganisms.

The specific biological reaction of the target organism is not analyzed directly in the exemplary processes described above and in many other processes. This makes efficient and effective control of the above processes often not very well possible.

Controlling or monitoring environmental conditions, such as (barometric) pressure, temperature, chemical concentration, moisture content and/or atmospheric humidity, acidity, light regime, noise and radiation in the environment surrounding us as well as in certain work rooms, such as clean-room facilities, surgery rooms, rooms for analytical measurements, storage rooms for chemicals, and the like is highly desired. From the viewpoint of safety, controlling or monitoring health-threatening chemical or biological substances in the environment surrounding us, including the air surrounding us, water masses, ground and food, is desired. Because the toxic components themselves are unknown or due to the fact that only specific combinations are undesired, the measurement of such substances is problematic in many cases, if not impossible. Thus, it is also desired that improved possibilities for controlling or monitoring environmental conditions become available.

In this context, the term environmental condition is meant to refer to the physical and/or chemical condition of the environment in a broad sense. More specific, in this context, it concerns the presence of specific chemicals in the environment.

Nowadays, a large variety of environmental conditions can be easily measured with instruments specially developed for this purpose. Also, specific biosensors for measuring a number of chemical compounds are available. However, many chemical compounds cannot be measured easily or not at all.

For instance, some microbial sensors for measuring specific chemicals are known from the literature. Tauriainen et al. (Wat. Res. 2000, Vol. 34, pp. 2661-2666) describe a microbial sensor for measuring metals such as cadmium, mercury, lead and arsenic. The microbial sensor is formed by E. coli, B. subtilis or S. aureus cells in which the expression of an inserted luciferase gene is under the control of a metal-sensitive regulatory unit. Belkin et al. (Wat. Res. 1997, Vol. 31, pp. 3009-3016) describe, for instance, a microbial sensor for measuring (toxic) substances and environmental stress. Such methods comprise the measurement of bioluminescence from recombinant bacterial cells where the induction route or the manner in which the organism reacts to a specific environmental condition is known.

The international patent application WO 99/09202 describes a method for detecting toxic substances in the environment, wherein the presence of toxic substances in the environment is determined by detection of beta-galactosidase protein as a result of the expression of a reporter gene in Dictyostelium cells. In this case as well, the manner in which the change in the organism is effected is known.

In many cases, however, the induction route is completely unknown, so using the methods according to the state of the art, only a small number of different environmental conditions can be determined.

Now it has surprisingly been found that microorganisms can be used as a measuring instrument because, due to change in biochemical composition, they can react to their environment in such a manner that, in principle, a large number of environmental conditions can be deduced therefrom, in principle without it being necessary that the induction routes that lead to the change are known.

The present invention provides a method for determining an environmental condition by measuring a biochemical composition of one or more microorganisms that are exposed to said environmental condition. Preferably, a natural biochemical composition is measured, as opposed to a recombinant biochemical composition.

The present invention also provides a method for determining changes in an environmental condition by measuring changes in a biochemical composition of one or more microorganisms that are exposed to said changes in an environmental condition.

The present invention further provides a method for determining an environmental condition comprising the measurement of a biochemical composition of one or more microorganisms that are exposed to said environmental condition, comparing the biochemical composition to a predetermined calibration line of a plurality of biochemical compositions of one or more microorganisms obtained through exposure of said one or more microorganisms to a plurality of environmental conditions and determining the environmental condition on the basis of the position of the biochemical composition on the calibration line.

The improved method enables monitoring and control of processes on the basis of intrinsic changes in microorganisms, whether or not already present in a process or a (process) environment, or introduced therein for the purpose of determining an environmental condition by means of a method according to the present invention.

The improved method provides the possibility to measure very small changes in the environment which were not measurable before.

The present invention uses the principle that a microorganism reacts strongly to outside influences. The intrinsic changes in the microorganism after applying an external stimulus consist in changes in amounts and nature of biomolecules such as RNA, protein and metabolites. It has now been found that the sum total of such changes is characteristic of the nature of the external stimuli that the microorganism receives. Further, by measuring and comparing concentrations of these biomolecules in the microorganisms under various conditions, groups of biomolecules can be selected that specifically change as a result of a reaction of the microorganism under the influence of a specific stimulus or environmental condition.

In this manner, microorganisms can be used as a specific sensor to determine environmental conditions, to determine the degree of change in these environmental conditions, to determine the nature and level of the stimulus and to make it possible to measure the effects of a diversity of stimuli on various processes and thus control these processes.

Examples of such a method include:
determining effects of temperature, or of temperature-based preserving methods, on the growth and/or death of microorganisms;
research into the effects of the nature of changes in process characteristics on the productivity of microorganisms;
microbial decomposition of undesired toxic substances (biodegradation);
testing the effectiveness of anti-microbial substances, such as antibiotics and biocides, on undesired growth of microorganisms and control of resistant bacteria;
testing the effects of different materials on the prevention of undesired growth of microorganisms on surfaces.

A method according to the present invention provides inter alia the possibility to determine the effect of antibiotics on microorganisms, which makes it possible to set up a screening assay for antibiotics or to evaluate the effect of specific treatment processes on the formation of, for example, biofilms and to improve it where needed. This analysis and control of biofilm formation is relevant for, for instance, the prevention of Legionella infection.

Also, fermentation processes and bioconversion processes can be controlled and improved in this manner. Furthermore, it is possible to develop an integrated system for made-to-measure process control for food preparation or new, mild preserving strategies for the production of, for instance, safe food products having a long storage life.

Also, environmental conditions, such as the occurrence of chemicals in an environment, can be determined by determining a specific stimulus-induced change in the composition of one or more microorganisms, by means of a method according to the present invention.

Microorganisms that can be used in a method according to the present invention are inter alia bacteria, archaea, fungi, yeasts, protozoa and algae. Preferably, bacteria, yeasts and/or fungi are used in a method according to the invention.

The microorganisms can be used in the form of a pure culture or monoculture of a single microorganism, but it is also suitable to use mixtures of microorganisms, including natural mixed populations and artificially composed mixed populations, in embodiments according to the present invention. It is also possible to use monocultures or mixed populations that occur naturally or in the process in one environmental condition in a totally different environmental condition.

In the control of processes in which specific microorganisms play a role, preferably the specific microorganisms from these processes will be used in a method according to the invention.

If a process takes place in the absence of microorganisms, microorganisms can be exposed to the process conditions for a shorter or longer period, for instance by introducing them into the processing room preferably from the start of the process, or by bringing (a part of) the process matter into contact with a microorganism outside the processing room.

If required, the microorganisms can be grown in advance. In certain embodiments, it is preferred to use the microorganisms in a uniform or standardized or defined physiological condition. This can, for instance, be the case if the microorganisms are exposed to the process conditions outside the processing room, for instance by exposing them to (a part of) the process matter, such as a potential antibiotic or samples of water, air, food, or when microorganisms are exposed to certain process conditions for a short or long period with the aim to determine the change of the microorganisms. Growing the microorganisms to a uniform or standardized or defined physiological condition is applicable in the cases referred to here.

In this context, a uniform or standardized or defined physiological condition is meant to refer to a condition of the microorganism that provides a known biochemical composition. If such a biochemical composition can be obtained repeatedly, it can be considered to be a standard.

It is not required, however, that the microorganisms are used in a uniform or standardized or defined physiological condition. In fact, measuring a change in the biochemical composition of microorganisms used in a method according to the present invention is sufficient to determine the conditions of the environment or changes therein, as long as this change in the biochemical composition of the microorganisms can be related to a specific environmental condition.

In a preferred embodiment, a method according to the invention comprises a comparison between at least two environmental conditions, namely a standard condition and an experimental condition, as a result of which the observed change in a biochemical composition of a microorganism will be substantially relative in nature.

A biochemical composition of microorganisms is meant, in the present context, to refer to a collection of test results which are the outcome of measurements on biomolecules of microorganisms used in a method according to the present invention. In particular, a biochemical composition of a microorganism is meant to refer to a plurality of different biomolecules of the microorganism.

Preferably, the measurements on biomolecules referred to are qualitative, but (semi-)quantitative measurements can also be used.

A suitable collection of measuring results is formed by the transcriptional expression profile of a microorganism that can be obtained by measuring the different messenger RNA molecules present in the cell (transcriptome). The plurality of different biomolecules that is measured to deduce an environmental condition therefrom comprises, in that case, a plurality of different RNA molecules. An alternative collection of measuring results is formed by the protein expression profile of a microorganism, which is obtained by measuring the different protein molecules present in the cell (proteome). Another alternative is measuring the collection of metabolites (metabolome).

The biochemical composition of a microorganism, which reflects the biological condition or status of a cell, can thus be very suitably measured on the basis of the transcriptional status, involving the determination of RNA quantities, the translational status, involving the determination of protein quantities, the activity status, involving the determination of the enzyme activities, or the status of metabolic routes, involving the determination of metabolite quantities, or by determining combinations thereof In fact, measurements on many different biomolecules can provide a biochemical composition of microorganisms that can be used in embodiments according to the present invention.

Such biomolecules can comprise polynucleotides such as nucleic acids, (poly)peptides or proteins, polysaccharides, lipids, lipopolysaccharides and other cellular macromolecules. In this context, also metabolic intermediates such as sugars, organic acids, alcohols, fatty acids, amino acids, nucleotides and the like can be measured. In embodiments according to the present invention, nucleic acids can very suitably be measured, such as RNA, including messenger, transfer and ribosomal RNA or combinations thereof

With great preference, in the present invention, the biochemical composition of a microorganism is determined by determining the transcriptional status of the cell, i.e. measured in the form of messenger RNA.

Measurements on biomolecules of microorganisms that can be used for forming a collection of measuring results that together provide a biochemical composition that can be used in embodiments according to the present invention in order to deduce an environmental condition therefrom are preferably in the form of a parallel biochemical analysis method.

Such measurements can, for instance, be carried out by separating the cellular biomolecules of microorganisms used according to the invention, marking them collectively or separately and detecting them qualitatively or quantitatively. Separating biomolecules, in this context, is meant to refer to the spatial separation of groups of identical molecules on the basis of their specific biochemical properties with the object to be able to detect and, if required, quantify a thus separated quantity of identical biomolecules. Spatially separating different groups of identical biomolecules is not always required, however. Marking biomolecules is meant, in this context, to refer to the specific or aspecific marking of biomolecules with specific markers with the object to detect and, if required, quantify the thus marked biomolecules.

For this purpose, use can be made of one or more techniques, such as:
in situ measuring techniques such as nucleic acid probe techniques and/or immunological measuring techniques; here, different cellular components can be measured separately, without the actual separation thereof taking place, by using specific or aspecific detection techniques, the suitability of which depends on the component to be detected;
electrophoretic and/or chromatographic measuring techniques; here, the cellular components can be separated on the basis of inter alia size, weight, charge, susceptibility to denaturation, after which detection takes place using specific or aspecific detection techniques, the suitability of which depends on the component to be detected;
microarray and/or biochip techniques; here, biochemical components are separated on the basis of affinity for a binding partner immobilized on a carrier and detection takes place using specific or aspecific detection techniques, the suitability of which depends on the component to be detected;

Suitable detection techniques that can be used in connection with above techniques are inter alia autoradiographic detection techniques, detection techniques based on fluorescence, luminescence or phosphorescence and chromogenic detection techniques. These techniques are well-known in the field of the detection of biomolecules.

Preferably, in the present invention, the transcriptional status of a microorganism is measured by using techniques involving hybridization to arrays of nucleic acid probes or nucleic acid-mimicking probes.

To determine a transcriptional status of a microorganism, the RNA, both the total RNA and the mRNA, can be isolated from one or more cells of the microorganism. Any RNA isolation method that does not select against the isolation of mRNA can be used in the isolation of such RNA (see, for instance, Ausubel et al., 1987-1993, Current Protocols in Molecular Biology, John Wiley & Sons. Inc. New York). Furthermore, large quantities of samples can be treated by using methods known to a skilled person, such as, for instance, the single-step isolation process of Chomczynski (1989, U.S., Pat. No. 4,843,155).

Transcripts (the RNA transcripts of DNA) represent RNA of differentially expressed genes, and can be identified in the different RNA samples by using a variety of methods known to a skilled person. For this purpose, use can be made of inter alia differential screening (Tedder et al., 1988. Proc. Natl. Acad. Sci. USA 85:208-212), subtractive hybridization (Hedrick et al., 1984, Nature 308:149-153; Lee et al., 1984, Proc. Natl. Acad. Sci. USA 88:2825), differential display (Liang & Pardee, 1992, Science 257:967-971; U.S. Pat. No. 5,262,311), expressed sequence tag (EST) (Adams et al., 1991, Science 52:1656), serial analysis of gene expression (SAGE) (Kinzler et al., U.S. Pat. No. 5,695,937) or cDNA AFLP (Vos et al., 1995, Nucleic Acids Res., 23, 4407-14) techniques.

The identification of such differentially expressed genes can further be used to design specific markers that enable accelerated determination of a specific environmental condition of a microorganism.

For measuring the different mRNA molecules in a microorganism, the mRNA is preferably first converted to complementary DNA (cDNA), to which purpose methods known to a skilled person are available, such as reverse transcription of the mRNA into cDNA using the enzyme reverse transcriptase. Optionally, the conversion of RNA to cDNA can be combined with an amplification of the cDNA by means of PCR (Mullis 1987, U.S. Pat. Nos. 4,683,195, 4,683,202, and 4,800,159)—optionally in a so-called “nested”, “multiplex” or “asymmetric” design—or using a ligase chain reaction (Barany 1991, Proc. Natl. Acad. Sci. USA 88:189-193; EP Application No. 320,308), a self sustained sequence replication (3SR) (Guatelli et al., 1990, Proc. Natl. Acad. Sci. USA 87:1874-1878), a strand displacement amplification (SDA) (U.S. Pat. Nos. 5,270,184, and 5,455,166), a transcriptional amplification system (Kwoh et al., Proc. Natl. Acad. Sci. USA 86:1173-1177), a Q-Beta Replicase (Lizardi et al., 1988, Bio/Technology 6:1197), Rolling Circle Amplification (RCA) or another method for the amplification of nucleic acids. In an alternative method, the RNA can also be used directly or first be amplified by means of RNA amplification methods available for this purpose, such as “Nucleic Acid Sequence-Based Amplification (NASBA)” (L. Malek et al., 1994, Meth. Molec. Biol. 28, Ch. 36, Isaac PG, ed., Humana Press, Inc., Totowa, N.J.) or by means of TMA.

Methods to obtain genetic information by using a nucleic acid array are known to a skilled person (see inter alia Chee et al., 1996, Science 274 (5287): 610-614).

In a method according to the present invention, the use of a DNA array is preferred. Such arrays of oligonucleotides comprise sequences that are specific to the genetic markers, and are also part of the present invention.

The manufacture of an oligonucleotide array according to the invention can be carried out using methods known to a skilled person. The manufacture and the use of solid carrier nucleic acid arrays for the detection of specific nucleic acid sequences has frequently been described (U.S. Pat. No. 5,571,639; Sapolsky et al., 1999, Genet. Anal.-Biomolecular Eng. 14, 187-192; Shena et al., 1995, Science 270, 467-470; Sheldon et al., 1993, Clinical Chem. 39, 718-719; Fodor et al., 1991, Science 251, 767-773).

A skilled person will capable of obtaining arrays after his own design and the corresponding array reading equipment from specialized suppliers (for instance Affymetrix Corp., Santa Clara, Calif., USA for DNA arrays and Ciphergen Biosystems, Fremont, Calif., USA for ProteinChip Array).

The determination of an environmental condition by measuring a biochemical composition of one or more microorganisms exposed to this environmental condition can be carried out as a single measurement. Also, the biochemical composition of microorganisms can be measured upon exposure to a plurality of environmental conditions in which one specific, defined environmental parameter is set at different values. In this manner, specific changes in the biochemical composition of these microorganisms as a result of this changing environmental parameter can be determined.

When measuring the biochemical composition of microorganisms exposed to a plurality of known environmental conditions in which one specific, defined environmental parameter is set at different values, it is possible to obtain calibration measurements by means of which a virtual calibration line can be drawn up, where one of the variables is formed by the different values at which the environmental parameter is set, and in which another variable is formed by the changing biochemical composition of microorganisms.

Such measurements can be analyzed using different analysis techniques, such as a statistical analysis, for instance a multivariate analysis, or other analysis techniques such as they presently exist or such as they can be developed for use in an embodiment according to the invention. In a method according to the present invention, use of analysis methods such as self-organizing maps, hierarchic clustering, multidimensional scaling, principal component analysis, supervised learning, k-nearest neighbors, support vector machines, discriminant analysis and partial least square methods are preferred. The analysis of the collection of measuring results that together describe a change in the biochemical composition of microorganisms as a result of changing, defined environmental conditions, produces a value for the environmental parameter and thus for the environmental condition.

In the determination of an unknown environmental condition by means of a method according to the invention, it is preferred to carry out the calibration measurements referred to prior to the determination of the unknown environmental condition. The measured biochemical composition of one or more microorganisms exposed to the unknown environmental condition then yields, on the basis of the position of the biochemical composition on the calibration line, a value for that environmental condition.

A calibration line as used in embodiments according to the present invention does not necessarily have to be considered an absolute calibration line. A virtual calibration line also finds suitable application in the invention. The combination of, for instance, different gene expression levels can be simply stored as results in a database, enabling comparison of the result of a measurement with the results of previous measurements. This virtual calibration line or database will thereby increase in size and detail, making the results increasingly well-founded on the basis of more and more new results.

### EXAMPLES

### Example 1

### Determination of Environmental Temperature by Measuring the RNA Expression of Pseudomonas putida

In the present test design, the Pseudomonas putida S12 strain was exposed to a temperature shock at different temperatures (35° C., 39° C. and 41° C.). The cells were fixed, after which RNA was isolated from the cells. The isolated RNA was hybridized on an array coated with genomic DNA fragments from Pseudomonas putida S12. The data analysis involved normalization and analysis according to principal component analysis (PCA). The test was carried out in duplicate and repeated once.

Bacterial Strain and Growth Conditions

Pseudomonas putida is an important decay organism that affects the quality of food products. From this viewpoint, this organism was selected. To simulate the composition of liquid food, the study was carried out in a culture medium that is rich in nutrients of trypton soy broth (TSB, Oxoid). Pseudomonas putida S12 pure culture was obtained by plating on Trypton Soy Agar (TSA, Oxoid) followed by incubation at 30° C. for 20 hours. After this, the strain was grown in TSB for 1 day at 30° C. in order to obtain a pre-accumulation.

Temperature Shock

P. putida S12 cultures that are in a specific physiological state as a result of temperature shock were obtained by diluting an overnight accumulation of P. putida S12 (35° C.) 20 times in 50 ml fresh TSB medium in Erlenmeyer flasks. In this manner, 4 simultaneous cultures of 50 ml were produced that were allowed approximately 7 hours to grow to an OD₆₆₀ of approximately 0.5 at 35° C. This was verified by measuring OD₆₆₀ of one culture every 30 minutes. At the moment the OD₆₆₀ was 0.5, the two other cultures were transferred to an incubation temperature of 39° C. and 41° C. respectively. The other culture remained incubated at 35° C. After 10 minutes, the cells from the three incubated cultures were quenched and harvested for RNA isolation.

RNA Isolation

Quenching of metabolic activity in the culture was achieved by spraying the 10 ml culture in a stirred solution of a cold 60% methanol solution in water that was kept at −40° C. (essentially as described by W. de. Koning & K. van Dam 1992. Anal. Biochem. 204:118-123). Then the cells were pelleted by 10-minute centrifugation at 4500 g at −20° C. The pellet was further treated at −45° C. or stored at −80° C. RNA was essentially isolated as described by Chin-Yi & Wilkins (1994. Anal. Biochem. 223:7-12). A 2-ml screw-cap Eppendorf tube was filled with 0.4 grams of zirconium beads and 500 μl Trizol (Invitrogen, GibcoBRL) at 0° C. The cell pellet (−45° C.) was resuspended in 450 μl borate buffer (Chin-Yi & Wilkins, 1994) at 37° C. and then added to the Eppendorf tube with beads and Trizol, mixed by hand and put on ice. The cells were broken up by beating them for 60 seconds in the Beadbeater (Biospec Products). The suspension was put on ice, after which 100 μl chloroform was added, vortexed and then centrifuged at 12,000 g at room temperature. The water phase was extracted using 500 μl phenol/chloroform (1/1) and then using chloroform with interim centrifugation in order to obtain a pure water phase. To the water phase 0.4 volumes of isopropanol were added and 0.4 volumes of 0.8M NaCitrate, 1.2M NaCl, mixed and cooled on ice for 10 min After that, RNA was pelleted by centrifugation at 20,000 g at 4° C. The pellet was washed with 70% ethanol of −20° C. and after centrifugation (20,000 g at 4° C.) dried under vacuum. The pellet was dissolved in 100 μl water.

Construction of Microarray

To make genome-wide analysis of gene expression in Pseudomonas putida S12 possible, a genomic bank of this organism was made. For this purpose, 50 micrograms of genomic DNA were incorporated in TE buffer with 20% glycerol and sheared for 30 seconds at 1 bar in a nebulizer. The average fragment size was estimated at 2-3 kb by means of gel electrophoresis. After precipitation, the ends were blunted in the presence of T4 DNA polymerase, Klenow polymerase and dNTPs. After heat inactivation of the enzymes, the ends were phosphorylized using T4 polynucleotide kinase. After heat inactivation, the mixture was again separated on an agarose gel and fragments between 2 and 3 kb were cut from the gel and were purified using a Qiagen gel extraction kit. The fragments obtained in this manner were ligated in vector pSMART according to the “Clone Smart Blunt cloning kit” protocol (Lucigen Corp.). A part of this ligation mixture was transformed in E. coli ElectroMax DH10B cells (Invitrogen) and plated on ampicillin plates. Using a colony picker, these colonies were picked and transferred into 96-well microtiter plates. After overnight growing at 37° C., glycerol was added to a final concentration of 15% and the glycerol stocks were stored at −80° C.

The genomic inserts from this bank were multiplied using PCR amplification in 96-well PCR plates. For this purpose, the following components were put together: 5 microliters 10× SuperTaq buffer; 5 microliters 2 mM dNTP (Roche Diagnostics); 1 microliter primer L1; 1 microliter primer R1; 37 microliters MQ water; 1 microliter glycerol stock; 1.5 U SuperTaq polymerase. Primer L1 has the following base sequence: 5′-CAG TCC AGT TAC GCT GGA GTC-3′. Primer R1 has the following base sequence: 5′-CTT TCT GCT ATG GAG GTC AGG TAT G-3′. Both primers contain a free NH2 group at the 5′-end followed by a C6 linker. The following PCR program was used for amplification: 4 min at 94° C.; 30× (30 sec at 94° C., 30 sec at 50° C., 3 min at 72° C.); 10 min at 72° C.; 4° C.

Following the reactions, the products were transferred to round-bottom 96-well plates and precipitated using isopropanol. The PCR products purified in this manner were transferred to 384-well plates, in which the DNA, after evaporating to dryness, was incorporated in 10 microliters 3×SSC. These spot plates were used to transfer a total of 5500 PCR products to microarray slides on which they were spotted in a regular and ordered pattern. After spotting, these microarrays were blocked, after which they were ready for further use in hybridization experiments.

Labeling RNA

For the fluorescent labeling of total RNA, 12.5 micrograms of RNA were used. To this RNA, 0.5 microliters RNAsin, 1.25 micrograms of random primer (p(dN)6; Roche Diagnostics) and water were added to a final volume of 5 microliters. This mixture was incubated for 10 min at 70° C., then for 10 min at 20° C. and then put on ice. Then the following components were added: 0.5 microliters RNAsin, 3 microliters 5× first strand buffer (SuperScriptII; Life Technologies), 1.5 microliters 0.1 M DTT, 3 microliters nucleotide mix (2.5 mM of dATP, dCTP, dGTP, 1 mM dTTP), 1 microliter Cy5-dUTP or Cy3-dUTP (Amersham Biosciences) and 1 microliter SuperScriptII enzyme (Life Technologies). After mixing, the reaction components were incubated for 2 hours at 42° C. Then the RNA was degraded by heating the samples for 2 min at 95° C., briefly spinning them down, adding 1.5 microliters fresh 2.5M NaOH and incubating this for 10 min at 56° C. By addition of 1.46 microliters 2.5M HAc, the reaction mixture is neutralized. This reaction mixture is purified by purification over an Autoseq G50 column (Amersham Biosciences) according to the accompanying directions. After this purification, 1/10 part of the labeled material is kept apart for spectrophotometric analysis.

Prehybridization/Hybridization/Washing

In preparation for the hybridization, slides were laid in Petri dishes in 20 ml prehybridization solution (1% BSA, 5×SSC, 0.1% SDS, filtered through a 0.45-micrometer-filter) and rotated for 45 minutes at 42° C. Then the slide was dipped 5× in filter-sterilized milliQ water. Then the slide was washed again in pure milliQ water, after which the slide was dipped 5× in isopropanol. Then the slide was dried using a N₂ gun and was then ready for hybridization.

The samples labeled with Cy5-dUTP and with Cy3-dUTP that were compared in one hybridization were combined in one cup. To this, 4 microliters yeast tRNA (25 micrograms/microliter) were added, after which the mixture was evaporated to dryness. After drying, the pellet was incorporated in 30 microliters EasyHyb (Roche Diagnostics) and denaturated (95° C., 1.5 min) after spinning down briefly. The mixture was then pipetted onto the prehybridized slide, covered with a cover slip and introduced into a Corning Hybridization room and incubated overnight at 42° C.

After hybridization, the slide was removed from the hybridization room and directly introduced into 20 ml 1×SSC/0.2% SDS solution of 37° C. The slide was then transferred to a fresh 1×SSC/0.2% SDS solution of 37° C., the cover slip staying behind in the first washing solution. After vigorous agitation, the slide was transferred to a 0.5×SSC washing solution of 37° C. and again well agitated. Then the slide was transferred to a 0.2×SSC washing buffer and mixed for 10 min at 20° C. on a rotation platform at 300 rpm. After replacing the washing buffer, this step was repeated. Then the slide was dried by blowing away the residual liquid using a N₂ gun.

Scanning and Image Analysis

After washing, the slide was stored in the dark or directly used for scanning. A quick scan with a resolution of 30 micrometers led to a selection of optimal scanning setting (ScanArray 4000, Perkin Elmer). The optimal scan of the Cy5 and Cy3 signal took place at a resolution of 10 micrometers and the resulting images were stored electronically. The images corresponding to a slide were opened in the software package ImaGene (version 4.2, BioDiscovery Inc;). After placing the grid, which represents the spot pattern of the DNA samples on the slides, and spot recognition, signals and background were calculated per spot. The data obtained were stored in. electronic files and used for further data processing.

Data Pre-Processing

After calculation of M and A values (Roberts et al., 2000, Science, 287, 873-880; Dudoit et al., 2000, Statistics, UC Berkeley, Technical reports #578), the raw data were normalized using Loess normalization (Cleveland et al., 1991, Statistics and Computing, volume 1 (1) pp. 47-62), leaving the following spots out of account: 1) spots with overload (with signal intensity in one or both channels being greater than 64000); 2) spots with the background being greater than the signal; 3) reference spots; 4) spots that were assigned a different flag than 0 by the image analysis package.

In this experiment, only spots that were found to have a good signal for all experiments were taken into account. After Loess normalization, the individual array results were normalized to unity (sum of squares equal to 1).

Data Analysis

The total dataset was divided in a training set and a test set for validation purposes. Then the dataset was analyzed using principal component analysis (PCA), with mean centering as the selected scaling method. Comparable results can be obtained using other scaling methods or using other multivariate methods, whether or not using the information in which clusters the different samples are present.

Results

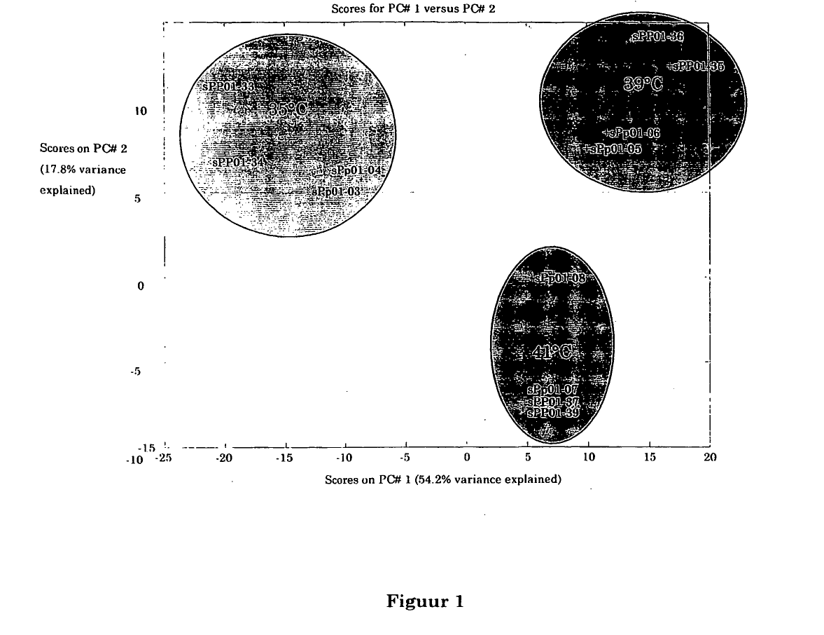

P. putida strain S12 was found to grow well or not in TSB depending on the incubation temperature. FIG. 1 shows the effect of the temperature on the growth curve. At 35° C., optimal growth was observed. At 41° C., there was hardly any growth. For measuring cell response under temperature stress, a comparison of the gene expression pattern on the microarray at 35° C. with those of 39° C. and 41° C. was chosen. For this purpose, 10 minutes after temperature shock, the cells were quenched, harvested and further processed for microarray analysis.

FIG. 1 shows a score plot in which the data sPP01-33 up through sPP01-39 represent the training set experiments and in which the data sPp01-03 up to and including sPp01-08 represent the test set experiments. The Figure shows a clear difference between the experiments carried out at the three temperature settings 35° C., 39° C. and 41° C. The two axes represent the principal components that together describe a total of 72% of the total variance in the dataset. The experiments Train-1 up to and including Train-6 from the training set were used to calculate a PCA model. The experiments Test-1 up to and including Test-6 were placed in the model, with FIG. 1 showing how the test dataset fits in the multivariate space as described using the training dataset. Here, it is clear to see that the points of the test set are in the right vicinity of the points of the training set. The differences between the repeats within both sets are smaller than the differences between the samples treated with other experimental conditions (temperature differences). The heating temperature of a medium could be determined by measuring the RNA expression (biochemical composition) of Pseudomonas putida present in the medium.

1. A method for determining an environmental condition by measuring a biochemical composition of one or more microorganisms exposed to said environmental condition.

2. A method for determining changes in an environmental condition by measuring changes in a biochemical composition of one or more microorganisms exposed to said changes in an environmental condition.

3. A method for determining an environmental condition comprising the steps of measuring a biochemical composition of one ore more microorganisms exposed to said environmental condition, comparing said biochemical composition to a predetermined calibration line of a plurality of biochemical compositions of said one or more microorganisms obtained by means of exposure of said one or more microorganisms to a plurality of environmental conditions and determining said environmental condition by means of the position of said biochemical composition on said calibration line.

4. A method according to claim 1, wherein said one or more microorganisms comprise bacteria, fungi and/or yeasts.

5. A method according to claim 1, wherein said biochemical composition comprises the transcriptome, the proteome and/or the metabolome of a microorganism.

6. A method according to claim 1, wherein said biochemical composition is the transcriptome.

7. A method according to claim 5, wherein said biochemical composition is determined using microarrays.

8. A method for controlling or monitoring an environmental condition, comprising a method according to claims 1.

9. A method for controlling a process, comprising a method according to claim 8.

10. Use of a method according to claims 1, wherein an environmental condition of a food preparation process, a biofilm formation process, a fermentation process and/or a bioconversion process is determined.

11. Use of a method according to claim 1, for determining a chemical and/or biological substance in air and/or aqueous environment.
